(19) United States
(12) Patent Application Publication
(10) Pub. No.: US 2026/0335006 A1
(43) Pub. Date: Oct. 29, 2026

(54) Intravascular Catheter Tip Electrode Assemblies

(71) Applicant: St. Jude Medical, Cardiology Division, Inc., St. Paul, MN (US)

(72) Inventors: Timothy G. Curran, St. Paul, MN (US); Gregory K. Olson, Elk River, MN (US)

(73) Assignee: St. Jude Medical, Cardiology Division, Inc., St. Paul, MN (US)

(21) Appl. No.: 19/678,559

(22) Filed: May 15, 2026

Related U.S. Application Data
(63) Continuation of application No. 17/256,551, filed on Sep. 23, 2019, now Pat. No. 12,653,607.
(60) Provisional application No. 62/738,473, filed on Sep. 28, 2018.

(51) Int. Cl. A61B 18/14; A61B 18/00

(52) U.S. Cl. CPC A61B 18/1492; A61B 2018/00351; A61B 2018/00577; A61B 2018/00839; A61B 2018/00875; A61B 2018/1435; A61B 2018/1467

(57) ABSTRACT

Aspects of the present disclosure are directed to intravascular electrophysiology catheters which utilize electrodes on flexible electronic circuit(s) to facilitate reduced assembly complexity and cost. An electrophysiology catheter system includes a catheter including a catheter shaft including a plurality of electrodes configured and arranged to sense electrophysiological characteristics of tissue and controller circuitry of the handle via communicatively coupled to the plurality of electrodes via flexible circuitry. The controller circuitry is configured and arranged to receive signals from the plurality of electrodes indicative of the electrophysiological characteristics of the tissue in contact with the plurality of electrodes. The flexible circuitry includes a plurality of internal electrical traces extending along one or more substrate layers and the plurality of internal electrical traces communicatively coupling the plurality of electrodes to the flexible circuitry. The catheter further includes an ablation electrode communicatively coupled to the flexible circuitry.

### CROSS-REFERENCES TO RELATED APPLICATIONS

The present application is a Continuation of U.S. Patent Appln. No. 17/256,551 filed Dec. 28, 2020 (Allowed); which is a U.S. National Stage 371 Appln. of PCT/IB2019/058052 filed Sep. 23, 2019; which claims the benefit of U.S. Provisional Appln. No. 62/738,473 filed Sep. 28, 2018; the full disclosures which are incorporated herein by reference in their entirety for all purposes.

### BACKGROUND OF THE INVENTION

Field. The present disclosure relates to the design and manufacture of intravascular catheters; more specifically, electrode assemblies for use in intravascular catheters.

Background Art. Electrophysiology catheters are used in a variety of diagnostic and/or therapeutic medical procedures to diagnose and/or correct conditions such as atrial arrhythmias, including for example, ectopic atrial tachycardia, atrial fibrillation, and atrial flutter. Arrhythmias can create a variety of conditions including irregular heart rates, loss of synchronous atrioventricular contractions and stasis of blood flow in a chamber of a heart which can lead to a variety of symptomatic and asymptomatic ailments and even death.

Medical procedures for diagnosing and/or treating arrhythmias may utilize an electrophysiology catheter deployed through a patient’s vasculature to a patient’s heart (or a chamber or vein thereof). The electrophysiology catheter carries one or more electrodes that can be used for cardiac mapping or diagnosis, ablation and/or other therapy delivery modes, or both. Once at the intended site, treatment may include radio frequency (RF) ablation, cryoablation, laser ablation, chemical ablation, high-intensity focused ultrasound-based ablation, microwave ablation, etc. The electrophysiology catheter may impart ablative energy to cardiac tissue to create one or more lesions in the cardiac tissue and oftentimes a contiguous or linear, and transmural lesion. This lesion disrupts undesirable cardiac activation pathways and thereby limits, corrals, or prevents stray/errant conduction signals that can form the basis for arrhythmias. As readily apparent, such diagnosis and therapy delivery requires precise diagnostic mapping and treatment.

While prior art basket catheters, such as the TOPERA FIRMap™ (now owned by Abbott Medical Systems) diagnostic cardiac catheter, have implemented spot electrodes on flexible printed circuitry to facilitate electrophysiology mapping of target tissue, the difficulties of implementing flexible circuit technology on the distal tip of linear electrophysiology (EP) catheters have resulted in the continued use of ring electrodes thereon.

### BRIEF SUMMARY OF THE INVENTION

The instant disclosure relates to intravascular catheter apparatus (and systems), and the manufacture thereof; more specifically, aspects of the present disclosure are directed to high-density mapping catheters and to map-ablate catheters for diagnosing and treating cardiac arrhythmias, for example. In particular, the instant disclosure relates to high density mapping/ablation catheters that include high-density mapping electrodes coupled to flexible circuitry which facilitate improved assembly efficacy and cost.

One embodiment of the present disclosure is directed to a distal tip assembly of an electrophysiology catheter. The distal tip assembly includes a catheter shaft, flexible circuitry, and a distal tip. The catheter shaft includes an outer surface with a trench extending into the outer surface. The flexible circuitry is inserted into the trench and coupled to the catheter shaft. The flexible circuitry includes one or more electrodes that sense electrophysiological characteristics of tissue. The distal tip is coupled to the distal end of the catheter shaft. In some embodiments, the electrodes are spot electrodes, and a top surface of the spot electrodes are flush with the outer surface of the catheter shaft. In various specific embodiments, the trench extends helically about the catheter shaft, the one or more spot electrodes are spaced along a length of the flexible circuitry, and thereby are circumferentially and longitudinally distributed about the catheter shaft.

Aspects of the present disclosure are directed to an electrophysiology catheter system. The system including a catheter and controller circuitry. The catheter includes a catheter shaft with a trench extending into an outer surface thereof, a distal tip coupled to the distal end of the catheter shaft, a catheter handle coupled to a proximal end of the catheter shaft, and flexible circuitry inserted into the trench and coupled to the catheter shaft. The flexible circuitry includes one or more electrodes that sense electrophysiological characteristics of tissue. The controller circuitry is communicatively coupled to the electrodes via the flexible circuitry. The controller circuitry receives signals from the electrodes indicative of the electrophysiological characteristics of tissue in contact with the electrodes. In more specific embodiments, the flexible circuitry includes a plurality of internal electrical traces extending along one or more substrate layers. The traces communicatively couple the electrodes to the controller circuitry. The electrodes are ring electrodes which surround at least a portion of the catheter shaft, and are electrically coupled with at least one of the plurality of electrical traces on the flexible circuitry. The catheter further includes a biocompatible outer covering extending over at least a portion of the flexible circuitry and the catheter shaft.

Yet further embodiments of the present disclosure are directed to a distal tip assembly of an electrophysiology catheter. The distal tip assembly includes a catheter shaft, flexible circuitry, and a distal tip coupled to a distal end of the catheter shaft. The flexible circuitry circumferentially extends about, and is coupled to, the catheter shaft. The flexible circuitry includes one or more spot electrodes that sense electrophysiological characteristics of tissue. In some embodiments, the flexible circuitry is coupled to the catheter shaft via a reflow process.

The foregoing and other aspects, features, details, utilities, and advantages of the present disclosure will be apparent from reading the following description and claims, and from reviewing the accompanying drawings.

### BRIEF DESCRIPTION OF THE DRAWINGS

Various example embodiments may be more completely understood in consideration of the following detailed description in connection with the accompanying drawings, in which:

FIG. 1 is a diagrammatic view of a system for performing diagnostic and/or therapeutic functions on cardiac tissue, consistent with various embodiments of the present disclosure;

FIG. 2 is an isometric front view of a partially-assembled electrophysiology catheter tip, consistent with various embodiments of the present disclosure;

FIG. 3 is an isometric front view of a partially-assembled electrophysiology catheter tip, consistent with various embodiments of the present disclosure;

FIG. 4 is an isometric front view of an assembled electrophysiology catheter tip, consistent with various embodiments of the present disclosure;

FIG. 5A is a side view of an electrophysiology catheter tip, consistent with various embodiments of the present disclosure;

FIG. 5B is a cross-sectional front view of the electrophysiology catheter tip of FIG. 5A, consistent with various embodiments of the present disclosure;

FIG. 6A is a side view of an electrophysiology catheter tip, consistent with various embodiments of the present disclosure;

FIG. 6B is a cross-sectional front view of the electrophysiology catheter tip of FIG. 6A, consistent with various embodiments of the present disclosure;

FIG. 7A is an isometric front view of an assembled electrophysiology catheter tip, consistent with various embodiments of the present disclosure;

FIG. 7B is an isometric front view of a partially-assembled electrophysiology catheter tip of FIG. 7A, consistent with various embodiments of the present disclosure;

FIG. 8A is an isometric front view of an assembled electrophysiology catheter tip, consistent with various embodiments of the present disclosure;

FIG. 8B is an isometric front view of a partially-assembled electrophysiology catheter tip of FIG. 8A, consistent with various embodiments of the present disclosure;

FIG. 9A is an isometric front view of an assembled electrophysiology catheter tip, consistent with various embodiments of the present disclosure;

FIG. 9B is an isometric front view of a partially-assembled electrophysiology catheter tip of FIG. 9A, consistent with various embodiments of the present disclosure;

FIG. 10A is an isometric front view of an assembled electrophysiology catheter tip, consistent with various embodiments of the present disclosure; and

FIG. 10B is an isometric front view of a partially-assembled electrophysiology catheter tip of FIG. 10A, consistent with various embodiments of the present disclosure.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

The instant disclosure relates to intravascular catheter apparatus (and systems), and the manufacture thereof; more specifically, aspects of the present disclosure are directed to high-density mapping catheters and to map-ablate catheters for diagnosing and treating cardiac arrhythmias, for example. In particular, the instant disclosure relates to high density mapping/ablation catheters that include high-density mapping electrodes coupled to flexible circuitry which facilitate improved assembly efficacy and cost.

In one embodiment of the present disclosure, a linear electrophysiology (“EP”) catheter includes a multi-layer flexible film printed circuit (also referred to as flexible circuitry) with a plurality of (spot) electrodes printed thereon. The flexible circuitry is coupled to a distal tip portion of the catheter. In some specific embodiments, the flexible circuitry may consist of Kapton® (polyimide), polyimide, polyetheretherketone, polyester, polyethylene terephthalate substrate, or a combination thereof. The flexible circuitry includes a plurality of internal electrical traces extending along one or more substrate layers, with the electrodes electrically coupled with at least one of the plurality of electrical traces (through a solder pad, vias, etc.).

The distal tip portion of the catheter shaft may include a trench which facilitates the flush assembly of the flexible circuit to the catheter shaft. This flush mounting of the flexible circuit and electrodes thereon minimizes possible thrombus formation, as well as mechanical irritation of cardiac tissue, for example. Additionally, the resulting smooth outer surface of the catheter shaft assists with passage of the catheter through an introducer (especially the hemostasis valve). Moreover, unlike prior art linear EP catheters which utilize ring electrodes, the catheter shaft of the present embodiment does not require apertures extending through the catheter shaft at each of the electrodes to facilitate communicative coupling with lead wires – which otherwise increase the potential for fluid ingress into a lumen of the catheter.

To facilitate biocompatibility of the flexible circuitry, the flexible circuitry may be plated/coated with a biocompatible material, such as gold. Overall size, width, pitch of the flexible circuitry may vary depending upon the given application.

The trench, in some embodiments, may be helically cut into an outer surface of the catheter shaft, and the flexible circuitry helically wound therein. Various known machining methodologies for forming the trench may be readily applied. For example, laser deposition may be utilized to remove material from the catheter shaft to form the trench which facilitates a width and thickness of the flexible circuitry. Alternatively, the trench may be machined, molded, reflowed, etc. During assembly of the catheter, the flexible circuit may be wound about the catheter shaft and coupled to the shaft within the trench utilizing adhesive, reflow, or other well-known coupling mechanisms. A distal most portion of the flexible circuit may be sandwiched between the catheter shaft and a distal tip of the catheter. In some embodiments, where the distal tip includes additional electrodes, the additional electrodes may be electrically coupled to the flexible circuit to facilitate communication of signals from the additional, distal tip electrodes to a proximal end of the catheter shaft. The distal most portion of the flexible circuit may enter an interior lumen of the catheter shaft at a bond between the catheter shaft and the distal tip. The flexible circuitry may then extend proximally to the handle, where electrical traces which extend from each of the electrodes terminate at an electrical connector. Such embodiments greatly reduce assembly complexity.

Aspects of the present disclosure are further directed to the use of spot electrodes, which often have smaller surface area then a ring electrode which circumferentially extends around a catheter shaft. Accordingly, spot electrodes have an inherent directionality and sensitivity. To mitigate the directionality and sensitivity of the spot electrodes in some embodiments, a distal tip of the EP catheter may include two flexible circuits which extend about the catheter shaft in a double helix arrangement or a single flexible circuit wound up and down the shaft twice. In such an embodiment, pairs of electrodes are positioned on the catheter shaft at equivalent longitudinal position, but radially offset about the longitudinal axis by approximately 180 degrees. While in some applications, electrode directionality may not be desirable; in others, sensing directionality of a wave front may be desirable to improve diagnosis and/or ablation therapy strategy.

Benefits of the present embodiment include lower cost and faster assembly as electrode stringing and electrode adhesive trimming are essentially eliminated. The use of spot electrodes instead of platinum (or platinum-iridium alloy) ring electrodes also reduces assembly cost. Moreover, in use, such an electrophysiology catheter limits the potential for thrombus formation as the contact surface of the spot electrodes are flush with an outer surface of the catheter shaft. Tight tolerance control of the trench depth on the catheter shaft may be critical to preventing thrombus when an ablation therapy is conducted by the EP catheter. The flush arrangement of the spot electrodes further facilitates a low potential for mechanical cardiac irritation. As the relative position of the electrodes along the catheter shaft are known by virtue of the pitch of the spiral and the linear spacing between each of the electrodes along the flexible circuit in some embodiments, more advanced implementations may utilize orientation independent sensing/omnipolar technology (“OIS/OT”) and related algorithms. OIS/OT and related algorithms are discussed in more detail in U.S. Provisional Appln. No. 61/944,426 filed Feb. 25, 2014, U.S. Appln. No. 15/118,522 filed Feb. 25, 2015 (now U.S. Patent No. 10,470,682), and international application no. PCT/US2014/011940 filed Jan. 16, 2014 (published as WO 2014/113612), which are hereby incorporated by referenced as though fully disclosed herein.

Known spacing between adjacent electrodes in an array (in two or more directions) facilitates simplified and robust OIS/OT assessments of orientation-specific electrical characteristics of myocardial tissue, for example. In some embodiments, known spacing permits 2-directional assessments of electrical activation direction and maximum voltage amplitude of sampled tissue. Moreover, known electrode spacing allows for the use of diagonal bipole pairs. Two diagonal bipole pairs, which are orthogonal relative to each other, measure the electrical characteristics of the same tissue region. The variation in readings between the orthogonal bipole pairs may be attributed to orientation-specific electrical characteristics of the contacted tissue. Embodiments of the present disclosure may further facilitate reduced complexity decimation by skipping intermediate electrodes, and forming bipole pairs with larger electrode spacing configurations than created by adjacent electrodes in the array. Decimation may be used to determine electrical characteristics of tissue at a less granular resolution. Further, a clinician may assess situational performance of the planar array at various bipole spacings. In various embodiments consistent with the present disclosure, adjacent bipole pairs may have various spacings, and be oriented in such a way as to facilitate various spatial orientations relative to one another.

The benefits of known electrode spacing along two or more perpendicular directions include a simplified computation of the electric field vector based only on average bipole voltages in the x, y directions. Known electrode spacing may also facilitate OIS/OT- methods that generate bipolar electrogram signals at various orientations with respect to wavefronts, so that a clinician may employ arbitrary catheter orientations (as discussed in more detail above). Finally, the known electrode spacing of the array facilitates a balanced and integrated view of voltage, fractionation, and/or activation patterns, which may be readily sampled from adjacent electrodes with known spacing. This information may then be used to compute a divergence and curl (i.e., to detect/locate foci and rotor cores from activation directions).

Various embodiments of the present disclosure are directed to EP mapping catheters, ablation catheters, and combinations thereof. In an example ablation catheter, a tip electrode may be communicatively coupled to controller circuitry via a dedicated lead wire, or integrated into a flexible circuit as a dedicated trace thereon. In some embodiments, an additional flexible circuit layer may be required for a wide trace which carries a desired ablation current with minimal/acceptable losses. In yet other implementations, a distal tip electrode may utilize an independent lead wire to deliver current for ablation therapy, but utilize a trace on the flexible circuit for sensing purposes (e.g., measuring electrograms or impedance).

In some simplified embodiments of the present disclosure, a trench need not be formed in an outer surface of the catheter shaft. Instead, the flexible circuit may be applied directly onto the outer surface of the catheter shaft, and heat-shrink tubing applied over the assembly. The reflow process of the heat-shrink tubing will (partially) compress the flexible circuit into the outer surface of the catheter shaft. To improve overall adhesion of the assembly, some specific embodiments may utilize polyurethane for the substrate of the flexible circuit (instead of, for example, polyimide), a base compound of Pebax® tubing (one type of heat-shrink tubing). Moreover, Pebax® is a common compound used for cardiac catheter shafts – further improving overall adhesion of the assembly. In such embodiments, it may still be desirable to have pre-formed grooves within the catheter shaft to facilitate improved placement of the flexible circuit along the catheter shaft. The pre-formed grooves correct for the majority of the depth adjustment between the spot electrodes and the outer surface of the catheter shaft, and reflow of the heat-shrink tubing corrects any slight height variations between the spot electrodes and the catheter shaft along the trench.

In yet other simplified embodiments of the present disclosure, flexible circuitry, including spot electrodes, may be directly coupled to a catheter shaft (absent a trench) via a reflow process and/or adhesive application.

Aspects of the present disclosure may further be applied to basket catheters, with one or more of the splines of the basket including flexible circuits, with spot electrodes mounted thereon, being helically wrapped around the splines. Such an embodiment facilitates the use of cylindrical splines which are less likely to cause trauma when moved within a cardiovascular system of the patient.

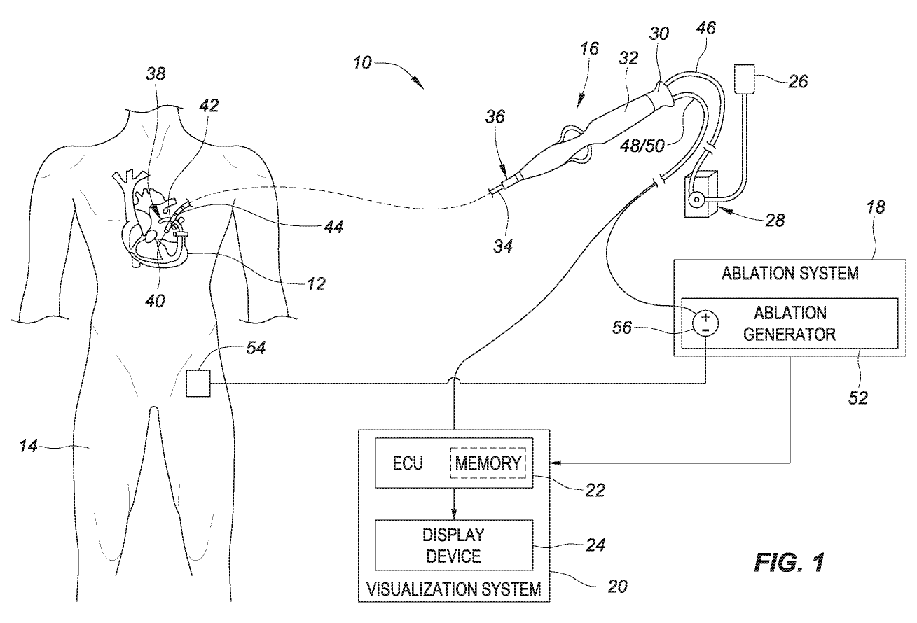

Referring now to the drawings wherein like reference numerals are used to identify identical components in the various views, FIG. 1 illustrates one example embodiment of a system 10 for performing one or more diagnostic and/or therapeutic functions in association with cardiac muscle 12 within a human body 14. It should be understood, however, that the system 10 may find application in connection with the ablation of a variety of other tissues within human and non-human bodies.

The system 10 may include a medical device (such as, for example, an electrophysiology catheter 16), an ablation system 18, and/or a system 20 for the visualization, navigation, and/or mapping of internal body structures. The system 20 may include, for example and without limitation, an electronic control unit (ECU) 22 and a display device 24. Alternatively, the ECU 22 and/or the display 24 may be separate and distinct from, but electrically connected to and configured for communication with, the system 20.

With continued reference to FIG. 1, the catheter 16 can be provided for examination, diagnosis, and/or treatment of internal body tissues such as within a cardiac muscle 12. In an example embodiment, the electrophysiology catheter 16 is a diagnostic catheter, such as a non-contact mapping catheter that may include a plurality of electrodes configured to monitor one or more electrical signals transmitted throughout the adjacent cardiac tissue. For example, electrophysiology catheter 16 may comprise a linear ablation catheter. The ablation catheter may be irrigated in an embodiment such that the catheter 16 may further comprise an inner fluid delivery tubing that may include at least one fluid delivery port. In the present embodiment, wherein the catheter 16 is an irrigated catheter, the catheter 16 can be connected to a fluid source 26 providing a biocompatible fluid such as saline, or a medicament, through a pump 28 (which may comprise, for example, a fixed rate roller pump or variable volume syringe pump with a gravity feed supply from the fluid source 26, as shown) for irrigation. It should be understood, however, that catheter 16 is not limited to a linear ablation catheter and is not limited to an irrigated catheter. Rather, in other embodiments, the catheter 16 may comprise an ablation catheter (e.g., radio frequency (RF), cryoablation, ultrasound, etc.), a mapping catheter, or a combination thereof. The catheter may be configured with or without irrigation.

In one example embodiment where the catheter comprises an ablation catheter, the catheter 16 is electrically connected to the ablation system 18 to allow for the delivery of ablative energy, or the like. The catheter 16 may include a cable connector or interface 30, a handle 32, a shaft 34 having a proximal end 36 and a distal end 38, and one or more electrodes 40, 42 mounted in or on the shaft 34 of the distal portion of catheter 16. In an example embodiment, the electrodes 40, 42 are disposed at or near the distal end 38 of the shaft 34, with the electrode(s) 40 comprising an ablation electrode disposed at the extreme distal end portion 38 of the shaft 34 (i.e., tip electrode 40), and the electrode(s) 42 comprising a spot electrode used, for example, with the visualization, navigation, and EP mapping system 20. Spot electrode(s) 42 can be configured to provide a signal indicative of both a position and orientation of at least a portion of the catheter 16. The catheter 16 may further include other conventional components such as, for example and without limitation, a temperature sensor (or sensors) 44, additional electrodes, and corresponding conductors.

The connector 30 provides mechanical, fluid, and electrical connection(s) for cables 46, 48, 50 extending from the pump 28, the ablation system 18, and the visualization, navigation, and/or mapping system 20. The connector 30 is conventional in the art and is disposed at the proximal end 36 of the catheter 16.

The handle 32 provides a location for the clinician to hold the catheter 16 and may further provide means for steering or guiding the shaft 34 within the body 14 as known in the art. Catheter handles 32 are generally conventional in the art and it will be understood that the construction of the handle 32 may vary. In an embodiment, for the purpose of steering the shaft 34 within the body 14, the handle 32 can be substituted by a controllable robotic actuator.

The shaft 34 is an elongate, tubular, flexible member configured for movement within the body 14. The shaft 34 supports, for example and without limitation, one or more electrodes (e.g., electrodes 40, 42), associated conductors, and possibly additional electronics used for signal processing, visualization, localization, and/or conditioning. The shaft 34 may also permit transport, delivery and/or removal of fluids (including irrigation fluids, medicaments, and bodily fluids, etc.), medicines, and/or surgical tools or instruments. The shaft 34 can include one or more lumens configured to house and/or transport electrical conductors, fluids, or surgical tools. The shaft 34 can be introduced into a blood vessel or other structure within the body 14 through a conventional introducer. The shaft 34 is then steered or guided through the body 14 to a desired location such as the tissue 12 with pullwires, tension elements, so-called push elements, or other means known in the art.

As generally illustrated in FIG. 1, an ablation system 18 can be comprised of, for example, an ablation generator 52 and one or more ablation patch electrodes 54. The ablation generator 52 generates, delivers, and controls ablation energy (e.g., radio-frequency) output by the ablation catheter 16 and the tip electrode 40 thereof, in particular. The generator 52 is conventional in the art and may comprise a commercially available unit sold under the model Ampere Cardiac Ablation Generator, available from Abbott Medical Systems. In one embodiment, the generator 52 may include an RF ablation signal source 56 configured to generate an ablation signal that is output across a pair of source connectors: a positive polarity connector SOURCE (+), which electrically connects to the tip electrode 40 of the catheter 16; and a negative polarity connector SOURCE(-), can be electrically connected to one or more of the patch electrodes 54. It should be understood that the term connectors as used herein does not imply a particular type of physical interface mechanism but is rather broadly contemplated to represent one or more electrical nodes (including multiplexed and de-multiplexed nodes). The source 56 is configured to generate a signal at a predetermined frequency in accordance with one or more user specified control parameters (e.g., power, time, etc.) and under the control of various feedback sensing and control circuitry. The source 56 may generate a signal, for example, with a frequency of about 450 kHz or greater for RF energy. The generator 52 may also monitor various parameters associated with the ablation procedure including, for example, impedance, the temperature at the distal tip of the catheter, applied ablation energy, power, force, proximity, and the position of the catheter, and provide feedback to the clinician or another component within the system 10 regarding these parameters.

The visualization, navigation, and/or EP mapping system 20 with which the electrodes 42 can be used may comprise an electric field-based system, such as, for example, ENSITE NAVX (aka EnSite Classic as well as newer versions of the EnSite system, denoted as ENSITE VELOCITY) and commercially available from Abbott Medical Systems and as generally shown with reference to U.S. Patent No. 7,263,397 titled “Method and Apparatus for Catheter Navigation and Location and Mapping in the Heart,” the entire disclosure of which is incorporated herein by reference. In accordance with an electric field-based system, the positioning electrode(s) 42 can be configured to be responsive to an electric field transmitted within the body of the patient. The electrode(s) 42 can be used to sense an impedance at a particular location and transmit a representative signal to an external computer or processor. In other embodiments, however, the visualization, navigation, and/or mapping system may comprise other types of systems, such as, for example and without limitation: a magnetic field-based system such as the CARTO System available from Biosense Webster, and as generally shown with reference to one or more of U.S. Patent Nos. 6,498,944 entitled “Intrabody Measurement,” 6,788,967 entitled “Medical Diagnosis, Treatment and Imaging Systems,” and 6,690,963 entitled “System and Method for Determining the Location and Orientation of an Invasive Medical Instrument,” the entire disclosures of which are incorporated herein by reference, or the gMPS system from MediGuide Ltd. of Haifa, Israel (now owned by Abbott Medical Systems), and as generally shown with reference to one or more of U.S. Patent Nos. 6,233,476 entitled “Medical Positioning System,” 7,197,354 entitled “System for Determining the Position and Orientation of a Catheter,” and 7,386,339 entitled “Medical Imaging and Navigation System,” the entire disclosures of which are incorporated herein by reference. In accordance with a magnetic field-based system, the electrode(s) 42 can be configured to be responsive to a magnetic field transmitted through the body of the patient. The electrode(s) 42 can be used to sense the strength of the field at a particular location and transmit a representative signal to an external computer or processor. The electrode(s) 42 may comprise one or more metallic coils located on or within the catheter 16 in a magnetic field-based system. Alternatively, a combination electric field-based and magnetic field-based system such as the CARTO 3 System also available from Biosense Webster, and as generally shown with reference to U.S. Patent No. 7,536,218 entitled “Hybrid Magnetic-Based and Impedance-Based Position Sensing,” the entire disclosure of which is incorporated herein by reference, may be used. In accordance with a combination electric field-based and magnetic field-based system, the electrodes 42 may comprise both one or more impedance-based electrodes and one or more magnetic coils. Commonly available fluoroscopic, computed tomography (CT), and magnetic resonance imaging (MRI)-based systems can also be used to facilitate visualization and navigation.

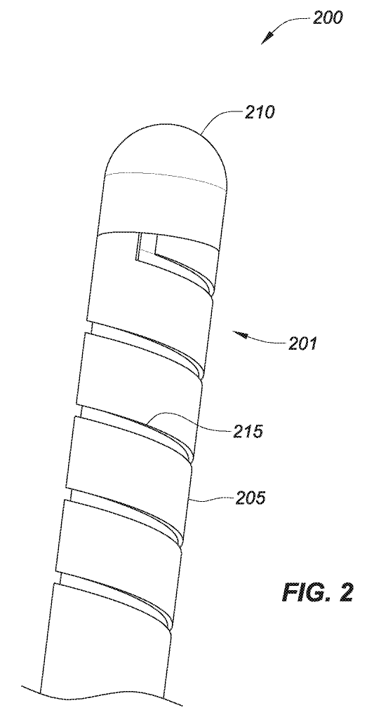

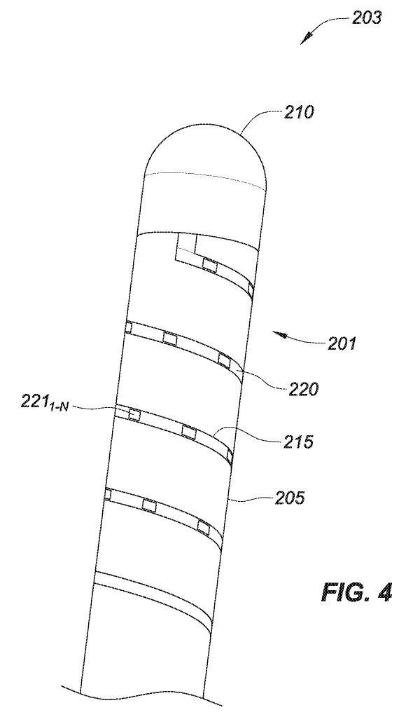

FIG. 2 is an isometric front view of a partially assembled electrophysiology catheter tip portion 200, consistent with various embodiments of the present disclosure. The partially assembled electrophysiology catheter tip portion 200 includes a catheter shaft 201 with a helical trench 215 cut into an outer surface 205 of a distal portion of the catheter shaft. The depth and width of the trench facilitates placement of a flexible circuit including spot electrodes thereon (as shown in FIG. 4). In some embodiments, the sensing surface of the spot electrodes is approximately flush with the outer surface 205 of the catheter shaft 201.

The catheter shaft 201 is coupled at a distal end to distal cap 210 (also referred to as a distal tip). The distal cap may be a non-traumatic tip via rounds thereon. The distal cap 210 may be coupled to the catheter shaft 201 using known methods, such as a press fit, adhesive, laser welding, ultrasonic, welding, among other known coupling techniques.

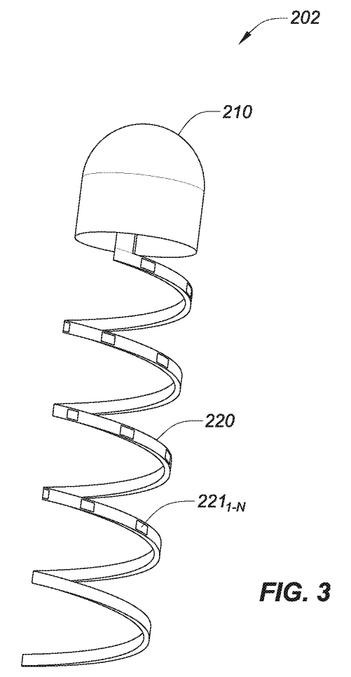

FIG. 3 is an isometric front view of a partially assembled electrophysiology catheter tip portion 202, consistent with various embodiments of the present disclosure. The partially assembled electrophysiology catheter tip portion 202 includes a flexible circuit 220 with a plurality of spot electrodes 221_{1-N} thereon, and a distal cap 210 coupled to a distal end of the flexible circuit 220. The partially assembled electrophysiology catheter tip portion 202 may be a sub-assembly that is prepared prior to final assembly of the electrophysiology catheter.

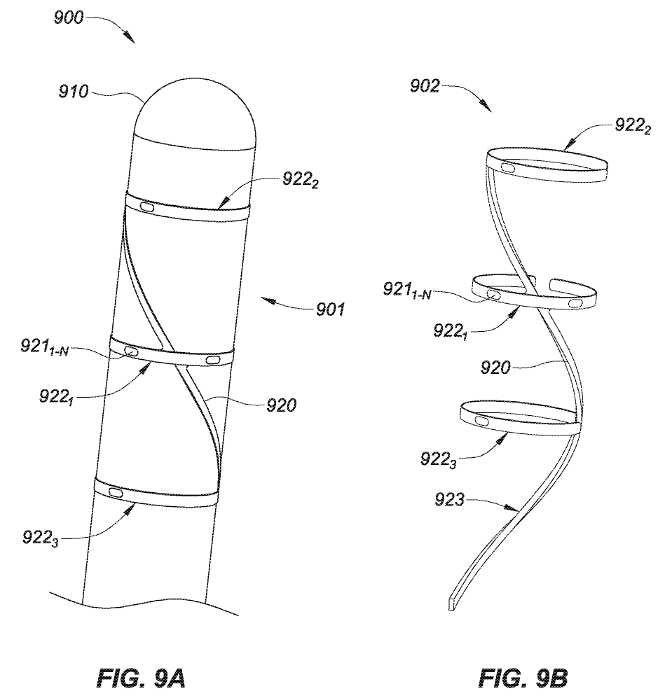

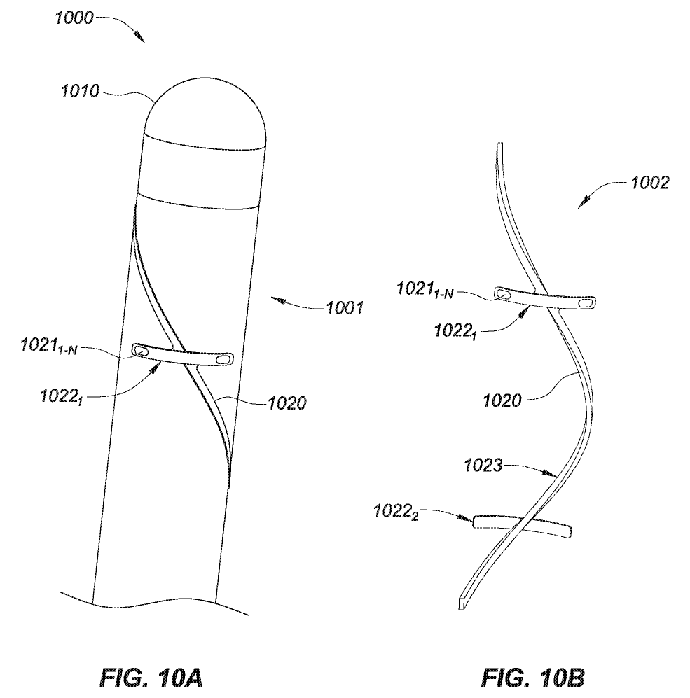

While FIG. 3 shows spot electrodes 221_{1-N} being evenly distributed along a length of the flexible circuit 220, various applications of the present application may utilize uneven electrode distributions or weighted distributions. For example, in some embodiments, it may be desirable to have a higher density of electrodes closer to the distal cap 210 of the catheter with a progressively smaller density of electrodes extending proximally. Moreover, as discussed above, in some embodiments it may be desirable to have a number of electrodes radially positioned about the catheter shaft at one or more longitudinal positions along the catheter shaft. In such an application, a single flexible circuit may be helically wound about the catheter shaft, two or more times, to position radially offset electrodes along the catheter shaft. Alternatively, or in addition, two or more distinct flexible circuits may be helically wrapped about the catheter shaft (e.g., in a double-helix fashion) to facilitate desired radial offsets of the electrodes. In yet other embodiments, a single flexible circuit may be wrapped entirely about a distal portion of the catheter shaft to facilitate numerous electrode configurations both radially and longitudinally along the shaft. In yet further embodiments, a wide-band flexible circuit may be helically wrapped about the catheter shaft with electrodes offset along a length and width of the flexible circuit to facilitate at least some radial offset of electrodes when assembled to the catheter shaft (see, e.g., FIGS. 9A-10B).

FIG. 4 is an isometric front view of an assembled electrophysiology catheter tip 203, consistent with various embodiments of the present disclosure. The assembled electrophysiology catheter tip portion 203 includes a catheter shaft 201, distal cap 210, and flexible circuit 220. As discussed in reference to FIGS. 2 and 3, the flexible circuit 220 is inserted into a helical trench 215 extending into an outer surface 205 of the catheter shaft. The depth of the trench, for some applications, may facilitate a plurality of electrodes 221_{1-N}, extending along a length of the flexible circuit, and being flush with or extending above the outer surface 205 of the shaft (for contact-type electrodes) or below the outer surface of the shaft (for non-contact-type electrodes). Further, in some embodiments, an additional biocompatible layer may be assembled over the electrophysiology catheter tip 203 and reflowed to prevent ingress of bodily fluids into the catheter and to mitigate sharp edges which may result in thrombus formation. In some applications, laser etching may be used to remove the biocompatible layer from above the electrodes (which may already comprise a biocompatible material, or be plated, i.e., gold plated).

When assembled, a distal end of flexible circuit 220 may extend into a lumen of the catheter shaft at the joint between catheter shaft 201 and distal cap 210. The flexible circuit may then be electrically coupled to lead wires which extend a length of the catheter shaft or extend itself to a proximal end of the catheter shaft. An electrical connector at a proximal end of the catheter may then complete an electrical circuit between controller circuity and electrodes 221_{1-N}.

To facilitate traditional printed circuit technology, the flexible circuits may be printed on a flat panel, with turns for where the flexible circuit will enter a lumen in the catheter shaft. The flat flexible circuit may then be wrapped around the catheter shaft to form the desired helical shape. Moreover, to facilitate long flexible circuits which are capable of extending a length of a catheter shaft (often 48” or longer), whereas existing printed circuit panel sizes are limited to 24”, the flexible circuit may be printed on the panel in a serpentine fashion and folded during assembly to achieve the desired lengths.

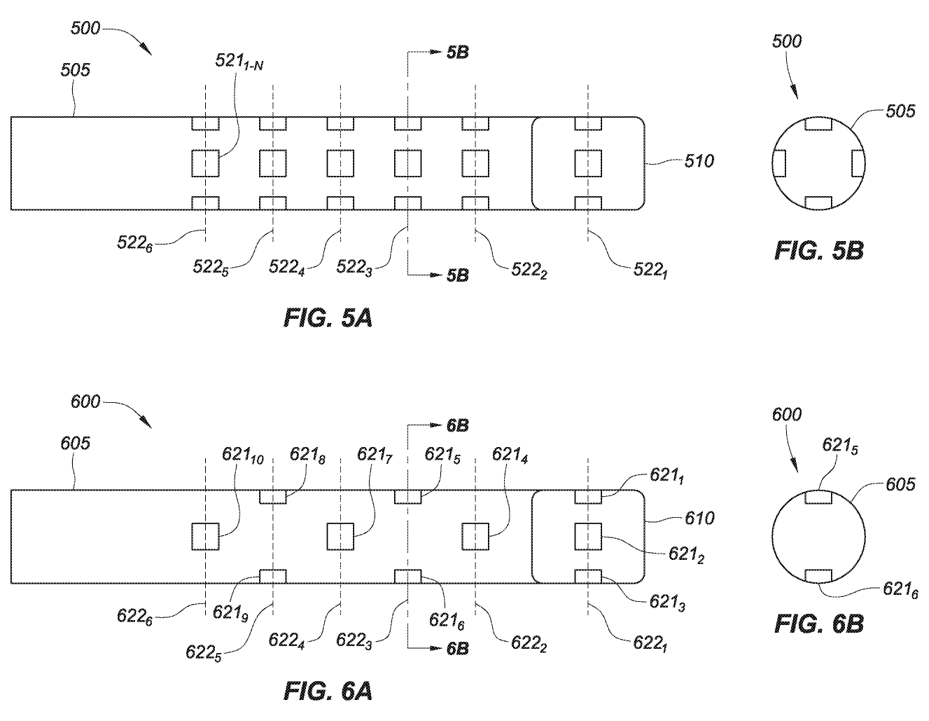

FIG. 5A is a side view, and FIG. 5B is a cross-sectional front view, of an electrophysiology catheter tip 500, consistent with various embodiments of the present disclosure. As shown in FIGS. 5A-B, the catheter tip 500 includes a catheter shaft 505 which distally extends to a distal cap 510. A plurality of electrodes 521_{1-N} are distributed radially and longitudinally about the catheter shaft 505. In the present embodiment, 4 electrodes are radially distributed around the catheter shaft (as shown in FIG. 5B) at each of planes 522₁₋₆. Each of the planes are positioned trans-axial relative to a longitudinal axis of the catheter shaft. As in various embodiments disclosed herein, such an electrode pattern may be implemented on the catheter shaft using one or more flexible circuits which are applied to the catheter shaft or otherwise integrated onto the catheter shaft itself. For example, through the application of conductive and non-conductive, and biocompatible layers on the catheter shaft itself.

FIG. 6A is a side view and FIG. 6B is a cross-sectional front view of an electrophysiology catheter tip 600, consistent with various embodiments of the present disclosure. As shown in FIGS. 6A-B, the catheter tip 600 includes a catheter shaft 605 which extends distally to a distal cap 610. A plurality of electrodes 621₁₋₁₀ are distributed radially and longitudinally about the catheter shaft 605. In the present embodiment, 2 electrodes are radially distributed about the catheter shaft (as shown in FIG. 6B) at each of planes 622₁₋₆. Each of the planes are positioned trans-axial relative to a longitudinal axis of the catheter shaft. The positioning of the electrodes at each respective plane are radially offset relative to electrodes on adjacent planes. As shown herein, the radial offset of electrodes at adjacent planes is approximately 180 degrees, though other embodiments are readily envisioned including 90 degrees, 270 degrees, etc. As in various embodiments disclosed herein, such an electrode pattern may be implemented on the catheter shaft using one or more flexible circuits which are applied to the catheter shaft or otherwise integrated onto the catheter shaft itself.

One particular advantage of the embodiment of FIGS. 6A-B is in regard to neutral surfaces (as discussed in more detail below). As the electrode pattern forms a T-shape along the surface of the catheter shaft, in response to steering inputs on the catheter shaft, the change in relative positioning of the electrodes due to the deflection of the catheter is minimal. This is particularly desirable in EP catheter applications utilizing OIS/OT algorithms which are dependent on known/fixed/predicable spacing of the electrodes for electrophysiology mapping, among other cardiac diagnostics.

In reference to the embodiments disclosed in FIGS. 5A-6B the catheter shaft 505/605 may transition between deflected and undeflected states. As shown in FIGS. 6A-B, electrodes may be distributed along a longitudinal axis of the catheter shaft 605 (e.g., 621₁₀ and 621₈). In the present embodiment the electrodes 621₁₋₁₀ are spot electrodes. In other embodiments, the catheter shaft can comprise a number of various quantities of electrodes. In yet other embodiments, the one or more electrodes disposed on the catheter shaft may comprise ring electrodes that extend (partially) around an outer circumference of the catheter shaft. In another embodiment, a ring electrode can be coupled to a portion of a conductor that is exposed to an exterior portion of the catheter shaft (i.e., solder pad on the flexible circuit). The ring electrode can comprise a composition that would be known to one of ordinary skill in the art and can be swaged, secured with adhesive, or otherwise coupled to the catheter shaft so that the ring is electrically coupled to the conductor. In yet other embodiments, the one or more electrodes can comprise electrodes that extend around a portion of an outer circumference of the shaft, or can comprise spot electrodes of different shapes and sizes than those illustrated in FIGS. 5A-6B. In yet another embodiment, the electrode can comprise a tab that is wrapped around an outer circumference of the catheter shaft and coupled thereto. In various embodiments, the tab may be configured to extend around the entire circumference or a partial circumference of an outer surface of the catheter shaft. The tab can comprise an electrically conductive material that has been added on to a base layer. While the illustrated embodiments show electrodes evenly spaced along the catheter shaft, in other embodiments the electrodes can be placed at various positions both axial and radial about the catheter shaft. By placing the electrodes at various axial and radial positions, separate and unique three-dimensional locations in space may be achieved and utilized as a centerline of the medical device for visualization in an imaging system, for example. Moreover, such an electrode configuration facilitates a neutral plane along a length of the catheter shaft with known and consistent catheter steering properties.

While the flexible circuits, and traces thereon, that communicatively couple electrodes 521_{1-N}/621₁₋₁₀ with controller circuitry at a proximal end of the catheter are not shown in FIGS. 5A-6B, the flexible circuits may be coupled to an exterior portion of catheter shaft 605 (as discussed throughout) or alternatively a portion of the catheter shaft may comprise a conductive material (e.g., traces) which extend proximally along the length of the catheter shaft. Once the electrodes are coupled to the traces, an insulative/bio-compatible material may be applied to the conductive material. To facilitate desirable steering characteristics, the traces and/or the flexible circuitry as a whole may run along a neutral plane of the shaft. The neutral plane of the catheter shaft can comprise a location along an outer diameter of the shaft that is approximately the same distance, in each direction, from a pullwire.

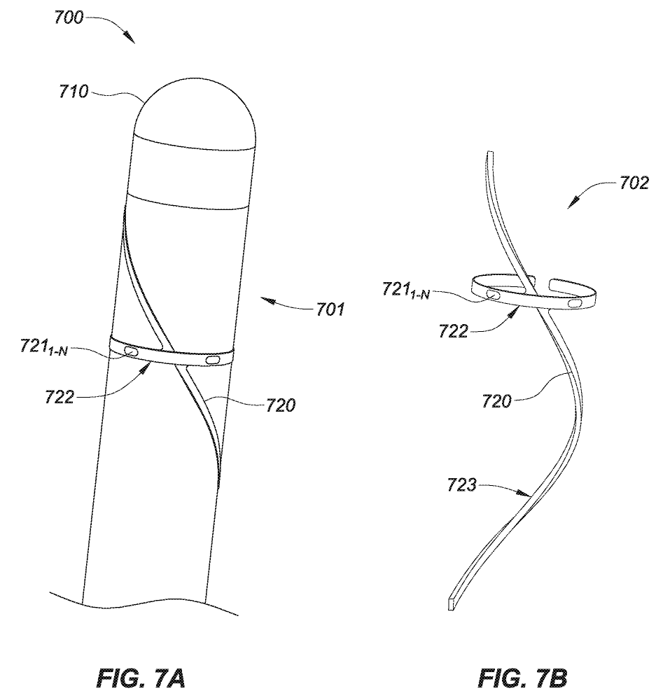

FIG. 7A is an isometric front view of an assembled electrophysiology catheter tip 700, and FIG. 7B is an isometric front view of a partially assembled electrophysiology catheter tip 702 of FIG. 7A. As shown in FIGS. 7A-B, the electrophysiology catheter tip includes a catheter shaft 701 coupled at a distal end to a distal cap 710, and flexible circuit 720. The flexible circuit includes both a helically extending portion 723 which extends longitudinally and helically about the catheter shaft 701, and a laterally extending portion 722 which extends about a majority of the catheter shaft circumference. The helically extending portion 723 includes a plurality of electrical traces that may be sandwiched between one or more circuit board layers of the flexible circuit, and which deliver electrical signals between a plurality of electrodes 721_{1-N} and controller circuitry at a proximal end of the catheter shaft. The laterally extending portion 722 includes the plurality of electrodes 721_{1-N} which may be (un)evenly distributed about the circumference of the catheter shaft 701. The plurality of electrodes 721_{1-N} may be positioned on an outer surface of the flexible circuit to facilitate contact-based electrophysiology sensing, positioned in a non-contact electrophysiology sensing manner by placing the electrodes beneath one or more layers of the flexible circuit, or a combination thereof. While the present embodiment depicts a single laterally extending portion 722, two or more laterally extending portions are readily envisioned.

The flexible circuit 720 may be inserted into a helically and laterally extending trench which extends into an outer surface of the catheter shaft 701. The depth of the trench for some applications may facilitate a plurality of electrodes 721_{1-N}, extending along a length of the flexible circuit, to be flush or to extend above an outer surface of the shaft (for contact-type electrodes) or below the outer surface of the shaft (for non-contact-type electrodes). Further, in some embodiments, an additional biocompatible layer may be assembled over the assembled electrophysiology catheter tip 700 and reflowed to prevent ingress of bodily fluids into the catheter and to mitigate sharp edges which may result in thrombus formation. In some applications, laser etching may be used to remove the biocompatible layer from above the electrodes (which may already comprise a biocompatible material, or be plated with a biocompatible layer, i.e., gold plated).

When assembled, a distal end of flexible circuit 720 may extend into a lumen of the catheter shaft at a joint between catheter shaft 701 and distal cap 710. The flexible circuit may then be electrically coupled to lead wires which extend a length of the catheter shaft or extend itself to a proximal end of the catheter shaft. An electrical connector at a proximal end of the catheter may then complete an electrical circuit between controller circuity and electrodes 721_{1-N}.

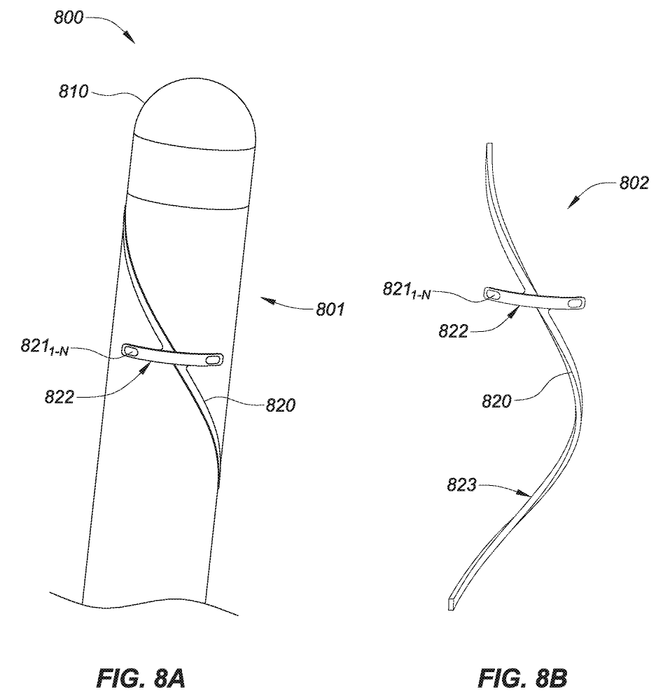

FIG. 8A is an isometric front view of an assembled electrophysiology catheter tip 800, consistent with various embodiments of the present disclosure, and FIG. 8B is an isometric front view of a partially assembled electrophysiology catheter tip 802 of FIG. 8A. As shown in FIGS. 8A-B the electrophysiology catheter tip includes a catheter shaft 801 coupled at a distal end to a distal cap 810, and flexible circuit 820. The flexible circuit includes both a helically extending portion 823 which extends longitudinally and helically about the catheter shaft 801, and a laterally extending portion 822 which extends about a small portion of the catheter shaft circumference (approximately one quarter of the circumference of the catheter shaft). The helically extending portion 823 includes a plurality of electrical traces that may be sandwiched between one or more circuit board layers of the flexible circuit, and which deliver electrical signals between a plurality of electrodes 821_{1-N} and controller circuitry at a proximal end of the catheter shaft. The laterally extending portion 822 includes the plurality of electrodes 821_{1-N} which may be (un)evenly distributed about the circumference of the catheter shaft 801. The plurality of electrodes 821_{1-N} may be positioned on an outer surface of the flexible circuit to facilitate contact-based electrophysiology sensing, positioned in a non-contact electrophysiology sensing manner by placing the electrodes beneath one or more layers of the flexible circuit, or a combination thereof. While the present embodiment depicts a single laterally extending portion, two or more laterally extending portions are readily envisioned. Furthermore, while the present embodiments depicts two electrodes, one or more electrodes are readily envisioned.

The flexible circuit 820 may be inserted into a helically and laterally extending trench which extends into an outer surface of the catheter shaft 801. The depth of the trench for some applications may facilitate a plurality of electrodes 821_{1-N}, extending along a length of the flexible circuit, to be flush or extending above an outer surface of the shaft (for contact-type electrodes) or below the outer surface of the shaft (for non-contact-type electrodes).

When assembled, a distal end of flexible circuit 820 may extend into a lumen of the catheter shaft at the joint between catheter shaft 801 and distal cap 810. The flexible circuit may then be electrically coupled to lead wires which extend a length of the catheter shaft or extend itself to a proximal end of the catheter shaft. An electrical connector at a proximal end of the catheter may then complete an electrical circuit between controller circuity and electrodes 821_{1-N}.

FIG. 9A is an isometric front view of an assembled electrophysiology catheter tip 900, and FIG. 9B is an isometric front view of a partially assembled electrophysiology catheter tip 902 of FIG. 9A. As shown in FIGS. 9A-B, the electrophysiology catheter tip includes a catheter shaft 901 coupled at a distal end to a distal cap 910, and flexible circuit 920. The flexible circuit includes both a helically extending portion 923 which extends longitudinally and helically about the catheter shaft 901 and laterally extending portions 922₁₋₃ which extend about a majority of the catheter shaft circumference. The helically extending portion 923 includes a plurality of electrical traces that may be sandwiched between one or more circuit board layers of the flexible circuit, and which deliver electrical signals between a plurality of electrodes 921_{1-N} and controller circuitry at a proximal end of the catheter shaft. The laterally extending portions 922₁₋₃ include the plurality of electrodes 921_{1-N} which may be (un)evenly distributed about the circumference of the catheter shaft 901. The plurality of electrodes 921_{1-N} may be positioned on an outer surface of the flexible circuit to facilitate contact-based electrophysiology sensing, positioned in a non-contact electrophysiology sensing manner by placing the electrodes beneath one or more layers of the flexible circuit, or a combination thereof. While the present embodiment depicts three laterally extending portions, one or more laterally extending portions are readily envisioned, depending on the resolution granularity desired for a given electrophysiology mapping application.

The flexible circuit 920 may be inserted into a helically and laterally extending trench which extends into an outer surface of the catheter shaft 901. The depth of the trench for some applications may facilitate a plurality of electrodes 921_{1-N}, extending along a length of the flexible circuit, to be flush, extend above an outer surface of the shaft (for contact-type electrodes), or extend below the outer surface of the shaft (for non-contact-type electrodes). Further, in some embodiments, an additional biocompatible layer may be assembled over the assembled electrophysiology catheter tip 900 and reflowed to prevent ingress of bodily fluids into the catheter and to mitigate sharp edges which may result in thrombus formation. In some applications, laser etching may be used to remove the biocompatible layer from above the electrodes (which may already comprise a biocompatible material, or plated biocompatible material, i.e., gold plated).

As shown in FIGS. 9A-B, three laterally extending portions 922₁₋₃ are distributed about a length of the catheter shaft 901. The placement of the electrodes 921_{1-N} radially about the catheter shaft may facilitate alignment, or as shown in FIGS. 9A-B, a radially offset arrangement between longitudinally adjacent electrodes.

While in FIGS. 9A-B, three laterally extending portions 922 are shown, a skilled artisan will appreciate that various numbers of laterally extending portions may be distributed along a length of the catheter shaft 901. Moreover, the distribution of the laterally extending portions may not necessarily be regular, but instead placement of the laterally extending portion may be weighted to locations along the catheter shaft where enhanced resolution electrophysiology mapping is desirable (e.g., near a distal tip 910 of the catheter shaft 901).

When assembled, a distal end of flexible circuit 920 may extend into a lumen of the catheter shaft at the joint between catheter shaft 901 and distal cap 910. The flexible circuit may then be electrically coupled to lead wires which extend a length of the catheter shaft or extend itself to a proximal end of the catheter shaft. An electrical connector at a proximal end of the catheter may then complete an electrical circuit between controller circuity and electrodes 921_{1-N}.

FIG. 10A is an isometric front view of an assembled electrophysiology catheter tip 1000, consistent with various embodiments of the present disclosure, and FIG. 10B is an isometric front view of a partially assembled electrophysiology catheter tip 1002 of FIG. 10A. As shown in FIGS. 10A-B, the electrophysiology catheter tip includes a catheter shaft 1001 coupled at a distal end to a distal cap 1010, and flexible circuit 1020. The flexible circuit includes both a helically extending portion 1023 which extends longitudinally and helically about the catheter shaft 1001 and laterally extending portions 1022₁₋₂ which extend about a small portion of the catheter shaft circumference (approximately one quarter of the circumference of the catheter shaft). The helically extending portion 1023 includes a plurality of electrical traces that may be sandwiched between one or more circuit board layers of the flexible circuit, and which deliver electrical signals between a plurality of electrodes 1021_{1-N} and controller circuitry at a proximal end of the catheter shaft. The laterally extending portions 1022₁₋₂ include the plurality of electrodes 1021_{1-N} which may be (un)evenly distributed about the circumference of the catheter shaft 1001. The plurality of electrodes 1021_{1-N} may be positioned on an outer surface of the flexible circuit to facilitate contact-based electrophysiology sensing, positioned in a non-contact electrophysiology sensing manner by placing the electrodes beneath one or more layers of the flexible circuit, or a combination thereof. While the present embodiment depicts a single laterally extending portion, two or more laterally extending portions are readily envisioned. Furthermore, while the present embodiments depicts two electrodes, one or more electrodes are readily envisioned.

The flexible circuit 1020 may be inserted into a helically and laterally extending trench which extends into an outer surface of the catheter shaft 1001. The depth of the trench for some applications may facilitate a plurality of electrodes 1021_{1-N}, extending along a length of the flexible circuit, to be flush with or extend above an outer surface of the shaft (for contact-type electrodes), or below the outer surface of the shaft (for non-contact-type electrodes).

When assembled, a distal end of flexible circuit 1020 may extend into a lumen of the catheter shaft at the joint between catheter shaft 1001 and distal cap 1010. The flexible circuit may then be electrically coupled to lead wires which extend a length of the catheter shaft or extend itself to a proximal end of the catheter shaft. An electrical connector at a proximal end of the catheter may then complete an electrical circuit between controller circuity and electrodes 1021_{1-N}.

As shown in FIGS. 10A-B, two laterally extending portions 1022₁₋₂ are distributed about a length of the catheter shaft 1001. The placement of the electrodes 1021_{1-N} radially about the catheter shaft may facilitate alignment, or a radially offset arrangement between longitudinally adjacent electrodes.

While in FIGS. 10A-B, two laterally extending portions 1022 are shown, a skilled artisan will appreciate that various numbers of laterally extending portions may be distributed along a length of the catheter shaft 1001. Moreover, the distribution of the laterally extending portions may not necessarily be regular, but instead placement of the laterally extending portion may be weighted to locations along the catheter shaft where enhanced resolution electrophysiology mapping is desirable (e.g., near a distal cap 1010 of the catheter shaft 1001).

In yet other embodiments consistent with FIGS. 7A-10B, laterally extending portion(s) of the flexible circuit may extend from a longitudinally extending portion of the flexible circuit, wrap circumferentially about the entire catheter shaft, and a distal end of the laterally extending portion(s) coupled back to the longitudinally extending portion of the flexible circuit. For example, the distal end of the laterally extending portion may be tucked under the longitudinally extending portion, or may be adhered with adhesive or another coupling mechanism well known in the art.

Various electrode patterns, especially linear electrode patterns extending along a catheter shaft and the use of neutral planes to facilitate improved catheter navigation within an intravascular system are generally shown with reference to PCT Application No. PCT/US2018/014430 titled “Sheath Visualization,” (published as WO 2018/136741) the entire disclosure of which is incorporated herein by reference.

While various embodiments discussed herein have been in reference to flexible circuitry, various other embodiments consistent with the present disclosure may utilize various types of stretchy conductors/substrates well known to those skilled in the art.

While aspects of the present disclosure have been presented as being readily applicable to radio-frequency ablation techniques, aspects of the present disclosure are also readily applied to irreversible electroporation (also referred to as direct current ablation). Moreover, while bipolar and monopolar RF techniques have been disclosed herein, variations on such techniques are also envisioned. For example, a bipolar ablation configuration may include alternating adjacent electrode polarities on the electrode array with the ground pad having a negative polarization. In one monopolar ablation configuration, the ground pad may have an alternating polarity over time, with adjacent electrodes carrying alternating polarities. Further, aspects of the present disclosure have been discussed including diagnosis and treatment of cardiac arrhythmias (e.g., atrial fibrillation); however, the present disclosure is readily applicable to the diagnosis and treatment of a number of different ailments, for example, Brugada syndrome.

Yet further embodiments consistent with the present disclosure may be directed to high-voltage direct current (“DC”) ablation (either bi-polar or mono-polar configuration). In such embodiments the high-voltage DC may include voltages between 400 and 4,000 Volts, and minimized current draw to target a voltage gradient rather than current delivery.

U.S. Provisional Appln. No. 62/414,634 filed Oct. 28, 2016, U.S. Provisional Appln. No. 62/572,186 filed Oct. 13, 2017, and U.S. Appln. No. 15/793,093 filed Oct. 25, 2017 (now U.S. Patent No. 11,172,858) are all generally directed to flexible, high-density mapping catheters and are incorporated by reference as though fully set forth herein.

While various embodiments of high-density electrode catheters are disclosed herein, the teachings of the present disclosure may be readily applied to various other catheter embodiments as disclosed, for example, in the following patents and patent applications which are hereby incorporated by reference: U.S. Provisional Appln. No. 61/753,429 filed Jan. 16, 2013; U.S. Provisional Appln. No. 60/939,799 filed May 23, 2007; U.S. Appln. No. 11/853,759 filed Sep. 11, 2007 (now U.S. Patent No. 8,187,267); U.S. Provisional Appln. No. 60/947,791 filed Jul. 3, 2007; U.S. Appln. No. 12/167,736 filed Jul. 3, 2008 (now U.S. Patent No. 8,206,404); U.S. Appln. No. 12/667,338 filed Jan. 20, 2011 (371 date – now U.S. Patent No. 8,827,910); U.S. Appln. No. 12/651,074 filed Dec. 31, 2009 (now U.S. Patent No. 8,979,837); U.S. Appln. No. 12/436,977 filed May 7, 2009 (now U.S. Patent No. 11,395,694); U.S. Appln. No. 12/723,110 filed Mar. 12, 2010 (now U.S. Patent No. 8,734,440); U.S. Provisional Appln. No. 61/355,242 filed Jun. 16, 2010; U.S. Appln. No. 12/982,715 filed Dec. 30, 2010 (now U.S. Patent No. 8,974,454); U.S. Appln. No. 13/159,446 filed Jun. 14, 2011 (now U.S. Patent No. 10,220,187); international Appln. No. PCT/US2011/040629 filed Jun. 16, 2011 (published as WO 2011/159861); U.S. Appln. No. 13/162,392 filed Jun. 16, 2011, published as U.S. 2012/0010490; U.S. Appln. No. 13/704,619 filed Dec. 16, 2012 (now U.S. Patent No. 10,118,015), which is a national phase of PCT/US2011/040781 filed Jun. 16, 2011 (published as WO 2011/159955 A1).

Various aspects of the present disclosure may be implemented in conjunction with OIS/OT-like signal processing algorithms for electrophysiology mapping. OIS/OT and related algorithms are discussed in more detail in U.S. Provisional Appln. No. 61/944,426 filed Feb. 25, 2014, U.S. Appln. No. 15/118,522 filed Feb. 25, 2015 (now U.S. Patent No. 10,470,682), and international Appln. No. PCT/US2014/011940 filed Jan. 16, 2014 (published as WO 2014/113612), which are hereby incorporated by referenced as though fully disclosed herein. Yet other embodiments of the present disclosure may be implemented in conjunction with various other electrophysiology mapping algorithms. For example, embodiments consistent with the present disclosure may utilize the electrode signal post-processing techniques, and electrophysiology mapping algorithms disclosed in the following publications, which are hereby incorporated by reference: Magtibay et al. JAHA 2017 (J Am Heart Assoc. 2017;6:e006447. DOI: 10.1161/JAHA.117.006447) (see, e.g., pages 6 and 7, and section titled “Omnipoles Provide the Largest Possible Bipolar Voltages”); and Haldar et al. Circulation AE 2017 (Circ Arrhythm Electrophysiol. 2017;10:e005018. DOI: 10.1161/CIRCEP.117.005018) (see, e.g., page 6, section titled “Omnipolar Voltage Amplitude Correlates to Largest Measurable Bipolar Vpp,” and FIG. 4).

Although several embodiments have been described above with a certain degree of particularity, those skilled in the art could make numerous alterations to the disclosed embodiments without departing from the spirit of the present disclosure. It is intended that all matter contained in the above description or shown in the accompanying drawings shall be interpreted as illustrative only and not limiting. Changes in detail or structure may be made without departing from the present teachings. The foregoing description and following claims are intended to cover all such modifications and variations.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Various modules or other circuits may be implemented to carry out one or more of the operations and activities described herein and/or shown in the figures. In these contexts, a “module” is a circuit that carries out one or more of these or related operations/activities (e.g., controller circuitry that receives and processes signals from the electrodes indicative of electrophysiology characteristics of the tissue). For example, in certain of the above-discussed embodiments, one or more modules are discrete logic circuits or programmable logic circuits configured and arranged for implementing these operations/activities. In certain embodiments, such a programmable circuit is one or more computer circuits programmed to execute a set (or sets) of instructions (and/or configuration data). The instructions (and/or configuration data) can be in the form of firmware or software stored in and accessible from a memory (circuit). As an example, first and second modules include a combination of a CPU hardware-based circuit and a set of instructions in the form of firmware, where the first module includes a first CPU hardware circuit with one set of instructions and the second module includes a second CPU hardware circuit with another set of instructions.

Certain embodiments are directed to a computer program product (e.g., nonvolatile memory device), which includes a machine or computer-readable medium having stored thereon instructions which may be executed by a computer (or other electronic device) to perform these operations/activities.

What is claimed is:

1. An electrophysiology catheter comprising:
a catheter shaft having a proximal end, a distal end, and an outer surface;
a distal tip assembly extending from a distal end of the catheter shaft;
flexible circuitry coupled to the distal tip assembly, the flexible circuitry comprising one or more electrodes configured and arranged to sense electrophysiological characteristics of tissue, wherein the flexible circuitry includes a plurality of internal electrical traces extending along one or more substrate layers, the plurality of internal electrical traces communicatively coupling the one or more electrodes to the flexible circuitry; and
an ablation electrode disposed at a distal end of the distal tip assembly and communicatively coupled to the flexible circuitry.

2. The catheter of claim 1, wherein the one or more electrodes comprise one or more spot electrodes.

3. The catheter of claim 1, wherein the catheter shaft comprises a trench extending into the outer surface and the flexible circuitry is disposed within the trench.

4. The catheter claim 3, wherein the trench extends helically about the catheter shaft, wherein the one or more electrodes are spaced along a length of the flexible circuitry.

5. The catheter of claim 3, wherein a top surface of the one or more electrodes are flush with the outer surface of the catheter shaft.

6. The catheter of claim 3, wherein the catheter shaft comprises a second helical trench radially offset from the trench, and the distal tip assembly further comprises a second flexible circuit comprising a plurality of spot electrodes coupled thereto, wherein the second flexible circuit is disposed within the second helical trench.

7. The catheter claim 1, wherein the one or more electrodes are further configured and arranged to facilitate electrophysiology mapping using orientation independent sensing/omnipolar technology.

8. The catheter of claim 1, further comprising a biocompatible layer that extends over the at least a portion of the flexible circuitry.

9. The catheter of claim 1, wherein the distal tip assembly further comprises a lead wire that is communicatively coupled to the ablation electrode and is configured and arranged to facilitate high-current ablation energy delivery to the ablation electrode.

10. The catheter of claim 1, wherein the one or more electrodes are disposed at or near the distal end of the catheter shaft.

11. The catheter of claim 1, wherein the one or more electrodes are ring electrodes disposed along a length of the flexible circuitry and electrically coupled to the flexible circuitry.

12. The catheter of claim 1, wherein the one or more substrate layers comprise one or more of polyimide, polyetheretherketone, polyester, polyethylene, and terephthalate material.

13. The catheter of claim 1, wherein the one or more electrodes are electrically coupled with at least one of the plurality of internal electrical traces through a solder pad.

14. The catheter of claim 1, wherein the one or more electrodes comprise a combination of electrophysiology mapping electrodes and impedance-based localization electrodes.

15. The catheter of claim 1, wherein the flexible circuitry is configured and arranged to circumferentially and longitudinally distribute the one or more electrodes along the catheter shaft.

16. The catheter of claim 13, wherein the flexible circuitry is coupled to the catheter shaft via a reflow process.

17. The catheter of claim 13, wherein the flexible circuitry is coupled to the catheter shaft via an adhesive.

18. An electrophysiology catheter system comprising:
a catheter comprising:
a catheter shaft comprising a proximal end and a distal end;
a catheter handle coupled to the proximal end of the catheter shaft;
a plurality of electrodes configured and arranged to sense electrophysiological characteristics of tissue;
controller circuitry of the handle communicatively coupled to the plurality of electrodes via flexible circuitry, the controller circuitry configured and arranged to receive signals from the plurality of electrodes indicative of the electrophysiological characteristics of the tissue in contact with the plurality of electrodes, wherein the flexible circuitry includes a plurality of internal electrical traces extending along one or more substrate layers, the plurality of internal electrical traces communicatively coupling the plurality of electrodes to the flexible circuitry; and
an ablation electrode communicatively coupled to the flexible circuitry.

19. The electrophysiology catheter system of claim 18, wherein the plurality of electrodes are electrically coupled with at least one of the plurality of electrical traces via a solder pad.

20. The electrophysiology catheter system of claim 18, further comprising at least one positioning electrode configured to sense a signal indicative of both a position and orientation of a distal portion of the catheter by sensing an impedance, the at least one positioning electrode communicatively coupled to the flexible circuitry.
